(19) United States
(12) Patent Application Publication
(10) Pub. No.: US 2025/0161626 A1
(43) Pub. Date: May 22, 2025

(54) SANITARY CATHETER DRAIN DEVICE

(71) Applicant: Jenna Schaal, Bayville, NJ (US)

(72) Inventor: Jenna Schaal, Bayville, NJ (US)

(21) Appl. No.: 18/904,905

(22) Filed: Oct. 2, 2024

Related U.S. Application Data
(63) Continuation-in-part of application No. 18/513,360, filed on Nov. 17, 2023.

(51) Int. Cl. A61M 25/00; A61M 39/22

(52) U.S. Cl. CPC A61M 25/0017; A61M 25/0041; A61M 25/0043; A61M 39/22; A61M 2025/0018; A61M 2205/0205; A61M 2209/088; A61M 2210/1085

(57) ABSTRACT

A sanitary catheter drain device includes a catheter tube having an insertion end configured for insertion into a user to establish fluid communication with a bladder of the user. A drain valve coupled to an open end of the catheter tube. The drain valve has a closed position preventing fluid passage through the drain valve and an open position permitting fluid passage from the open end of the catheter tube through the drain valve. A cap is couplable to the drain valve such that the cap covers an outlet of the drain valve.

### CROSS-REFERENCE TO RELATED APPLICATIONS

I hereby claim the benefit under 35 U.S.C., Section 120 of U.S. application Ser. No. 18/513,360 filed Feb. 21, 2024.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to catheter drain device and more particularly pertains to a new catheter drain device for facilitating drainage of a catheter and preventing infection through the catheter.

### (2) Description of Related Art Including Information Disclosed Under 37 CFR 1.97 and 1.98

The prior art relates to catheter drain devices. Known prior art provides for urine collection bags which must be attached to a person and detached for emptying of the urine collection bag. The prior art lacks a valved cap allowing for drainage of a catheter periodically without having to disengage the catheter tube. Further, an antiseptic pad is incorporated into a drain valve cap to disinfect and prevent contamination and infection through the catheter.

### BRIEF SUMMARY OF THE INVENTION

An embodiment of the disclosure meets the needs presented above by generally comprising a catheter tube having an insertion end configured for insertion into a user to establish fluid communication with a bladder of the user. A drain valve coupled to an open end of the catheter tube. The drain valve has a closed position preventing fluid passage through the drain valve and an open position permitting fluid passage from the open end of the catheter tube through the drain valve. A cap is couplable to the drain valve such that the cap covers an outlet of the drain valve.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a side view of a sanitary catheter drain device according to an embodiment of the disclosure.

FIG. 2 is a front view of an embodiment of the disclosure.

FIG. 3 is an exploded front view of an embodiment of the disclosure.

FIG. 4 is a cross-sectional view of an embodiment of the disclosure taken along line 4-4 in FIG. 3.

FIG. 5 is a front view of an embodiment of the disclosure.

FIG. 6 is a partial cutaway view of a drain valve an embodiment of the disclosure in a closed position.

FIG. 7 is a partial cutaway view of a drain valve an embodiment of the disclosure in an open position.

### DETAILED DESCRIPTION OF THE INVENTION

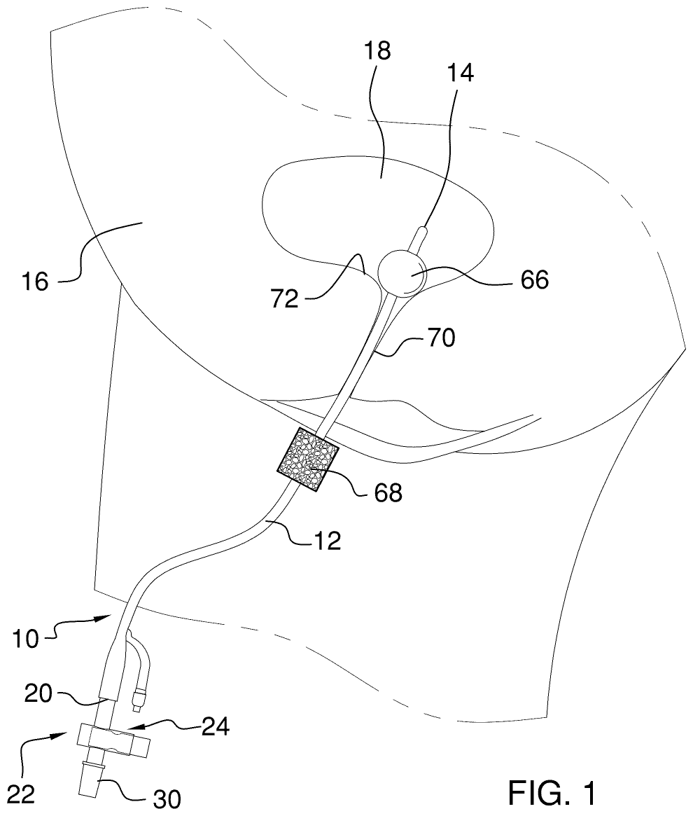

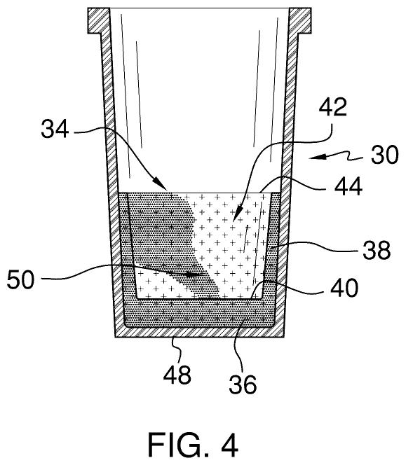

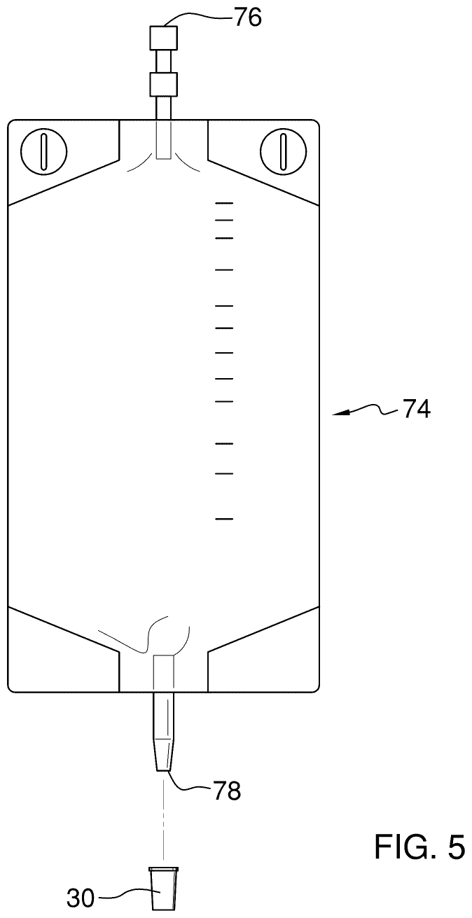

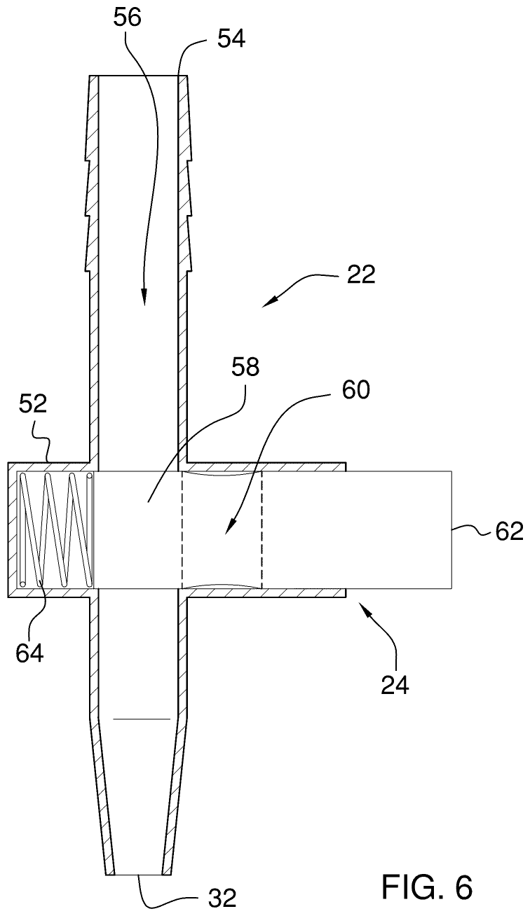

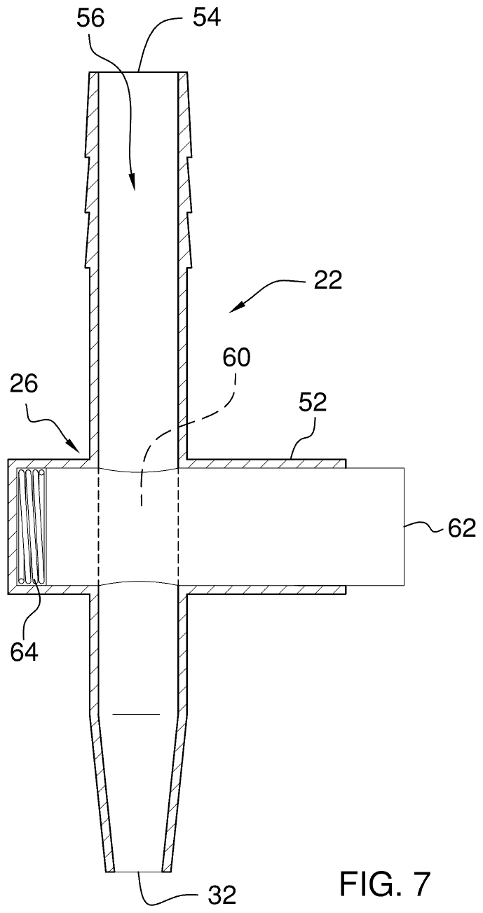

With reference now to the drawings, and in particular to FIGS. 1 through 7 thereof, a new catheter drain device embodying the principles and concepts of an embodiment of the disclosure and generally designated by the reference numeral 10 will be described.

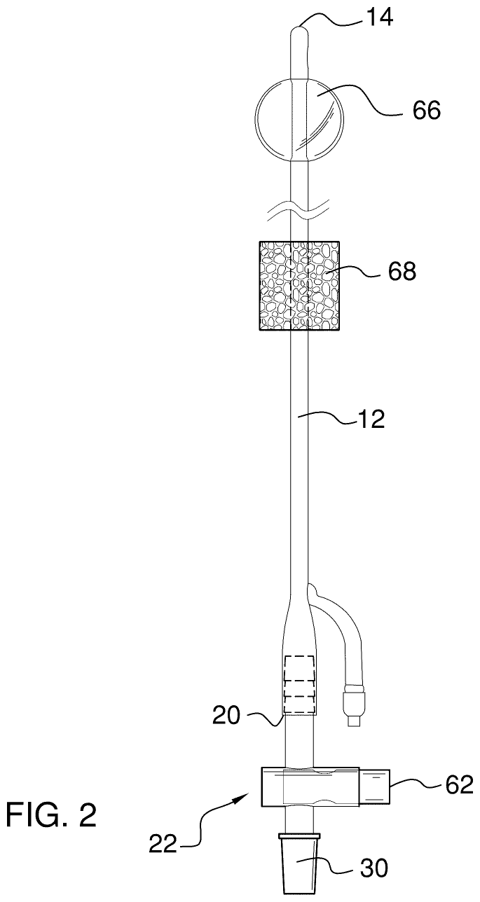

As best illustrated in FIGS. 1 through 6, the sanitary catheter drain device 10 generally comprises a catheter tube 12 having an insertion end 14 configured for insertion into a user 16 to establish fluid communication with a bladder 18 of the user 16. The catheter tube 12 has an open end 20 opposite the insertion end 14. A drain valve 22 is coupled to the open end 20 of the catheter tube 12 as shown in FIGS. 1 and 2. The drain valve 22 has a closed position 24 wherein the drain valve 22 prevents fluid passage through the drain valve 22 from the open end 20 of the catheter tube 12. The drain valve 22 has an open position 26 wherein the drain valve 22 permits fluid passage from the open end 20 through the drain valve 22. A cap 30 is couplable to the drain valve 22 such that the cap 30 covers an outlet 32 of the drain valve 22. A pad 34 is coupled to the cap 30 wherein the pad 30 is positioned to abut the outlet 32 of the drain valve 22 when the cap 30 is engaged to the drain valve 22. The pad 34 is constructed of an antimicrobial material which may be but is not limited to polyurethane foam, hemp balm, or gauze infused or otherwise treated with a known antimicrobial agent. Thus, the pad 34 will inhibit bacteria and potential infection passing through the user from the outlet 32 of the drain valve 22.

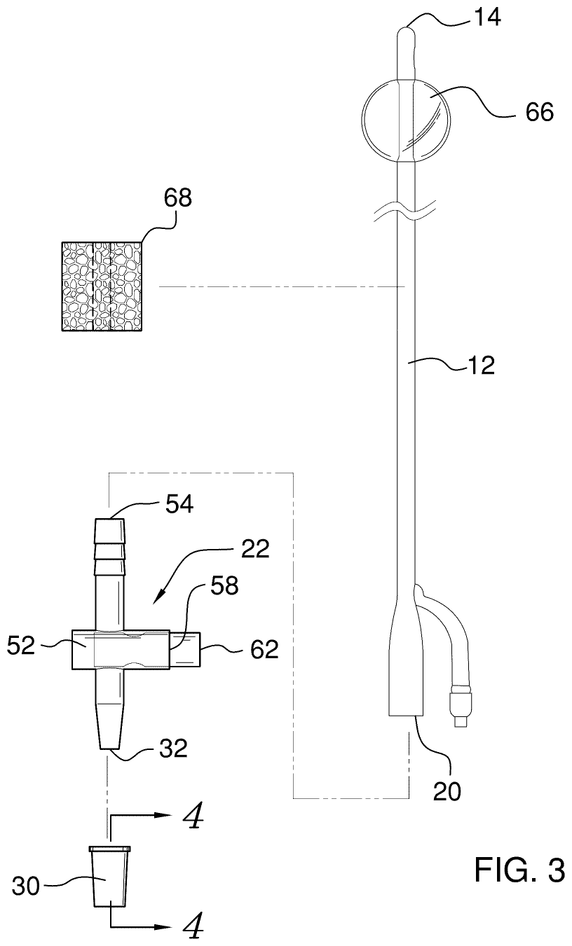

The pad 34 has a bottom wall 36 and a peripheral wall 38 coupled to and extending upwardly from a peripheral edge 40 of the bottom wall 36. Thus, the pad 34 forms a well 42 in the pad 34 over the bottom wall 36. The well 42 will receive the outlet 32 of the drain valve 22 when the cap 34 is engaged to the drain valve 22. The peripheral wall 38 tapers from the bottom wall 36 of the pad 34 to a distal edge 44 of the peripheral wall 38 relative to the bottom wall 36 of the pad 34 to promote snug engagement of the outlet 32 within the well 42. As can be seen in FIG. 3, the outlet 32 may taper such that the tapering of the peripheral wall 38 also helps to prevent snagging and bunching of the peripheral wall 38 as the outlet 32 is inserted into the well 42 because there is enhanced space for the tip of the outlet 32 to pass into an opening in the well at the distal edge 44. The pad 34 further has a height from a bottom edge 48 of the pad 34 to the distal edge 44 of the peripheral wall 38 less than half a length of the cap 30 wherein the pad 34 is positioned in a bottom half 50 of the cap 30 when fully inserted into the cap 30. The bottom wall 36 has a thickness greater than twice a maximum thickness of the peripheral wall 38 of the pad 34. This facilitates absorption into the pad 34 and cushions full insertion of the outlet 32 into the cap 34.

The drain valve 22 includes a housing 52 having the outlet 32 of the drain valve 22 and an inlet 54 insertable into the open end 20 of the catheter tube 12. A channel 56 extends through the housing 52 from the inlet 54 to the outlet 32. A plug 58 is coupled to the housing 52. The plug 58 extends through the channel 56. A duct 60 passes through the plug 58. The duct 60 is positionable to align with the channel 56 to define the open position 26 of the drain valve 22. The duct 60 is positionable offset from the channel 56 wherein the plug 60 obstructs the channel 56 to define the closed position 24 of the drain valve 22. A button 62 extends from the housing 52. The button 62 is coupled to the plug 60 wherein the plug 60 is configured for moving between the closed position 24 and the open position 26 when button 62 is pressed. The button 62 may be an integral portion or extension of the plug 60 extending from the housing 52. A biasing member 64 is positioned in the housing 52. The biasing member 64 biases the plug 60 such that the drain valve 22 is in the closed position 24 when the button 62 is not being pressed.

The elements of the invention as described above may be used with a balloon 66 coupled to the catheter tube 12 adjacent to the insertion end 14 wherein the balloon 66 is configured for positioning in the bladder 18 of the user 16 to inhibit the insertion end 14 from coming out of the bladder 18 of the user 16. A sponge 68 may be coupled to the catheter tube 12. The sponge 68 is spaced from the balloon 66 such that the sponge 68 is configured for positioning adjacent to a urethra 70 of the user 16 while the balloon 66 is positioned against a wall 72 of the bladder 18 of the user 16. Thus, the catheter tube 12 is configured to be restricted in movement relative to the user 16.

It is contemplated in place of the drain valve 22 the device 10 may incorporate a urine collection bag 74. The urine collection bag 74 has an inlet port 76 and an outlet port 78. The inlet port 76 is in fluid communication with the open end 20 of the catheter tube 12. It will be known to one of ordinary skill in the art that the drawing figures show structures as described herein which can be connected as described without explicitly being shown in the drawing figures. For example, connection of the inlet port 76 of the urine collection bag 74 to the open end 20 of the catheter tube 12. The cap 30 is couplable to the outlet port 78 such that the cap 30 covers the outlet port 78 of the urine collection bag 74. Further, it is contemplated that the disclosure herein further teaches and supports the connection of the urine collection bag 74 as described to be coupled between the catheter tube 12 and the drain valve 22 with the cap 30 coupled to the drain valve 22 as described above.

In use, the catheter tube 12 is inserted in a conventional manner into the user 16. The drain valve 22 is coupled either directly to the catheter tube or may be coupled to the urine collection bag 74 and catheter tube 12. The cap 30 may be coupled directly to the drain valve 22 or the urine collection bag 74. The drain valve 22 permits selective draining of urine from the catheter tube and provides a relatively small device to be concealed by the user 16 as opposed to using the urine collection bag 74. Whether used with or without the urine collection bag 74, the drain valve 22 facilitates release of urine. The cap 30 is selectively used to cover the outlet 32 of the drain valve 22 or the outlet port 78 of the urine collection bag 74 such that the pad 30 inhibits contamination and potential infection through the catheter tube 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sanitary catheter drain device comprising:
a catheter tube having an insertion end configured for insertion into a user to establish fluid communication with a bladder of the user, said catheter tube having an open end opposite said insertion end;
a drain valve coupled to said open end of said catheter tube, said drain valve having a closed position wherein said drain valve prevents fluid passage through said drain valve from said open end of said catheter tube, said drain valve having an open position wherein said drain valve permits fluid passage from said open end through said drain valve; and
a cap, said cap being couplable to said drain valve such that said cap covers an outlet of said drain valve.

2. The sanitary catheter drain device of claim 1, further comprising a pad, said pad being coupled to said cap wherein said pad is positioned to abut said outlet of said drain valve when said cap is engaged to said drain valve.

3. The sanitary catheter drain device of claim 2, further comprising said pad being constructed of an antimicrobial material.

4. The sanitary catheter drain device of claim 2, further comprising said pad having a bottom wall and a peripheral wall coupled to and extending upwardly from a peripheral edge of said bottom wall wherein said pad forms a well in said pad over said bottom wall.

5. The sanitary catheter drain device of claim 4, further comprising said peripheral wall tapering from said bottom wall of said pad to a distal edge of said peripheral wall relative to said bottom wall of said pad.

6. The sanitary catheter drain device of claim 5, further comprising said pad having a height from a bottom edge of said pad to said distal edge of said peripheral wall less than half a length of said cap wherein said pad is positioned in a bottom half of said cap when fully inserted into said cap.

7. The sanitary catheter drain device of claim 4, further comprising said bottom wall having a thickness being greater than twice a maximum thickness of the peripheral wall of said pad.

8. The sanitary catheter drain device of claim 1, wherein said drain valve further comprises:
a housing having said outlet of said drain valve and an inlet insertable into said open end of said catheter tube;
a channel extending through said housing from said inlet to said outlet;
a plug coupled to said housing, said plug extending through said channel, a duct passing through said plug, said duct being positionable to align with said channel to define said open position of said drain valve, said duct being positionable offset from said channel wherein said plug obstructs said channel to define said closed position of said drain valve.

9. The sanitary catheter drain device of claim 8, further comprising a button extending from said housing, said button being coupled to said plug wherein said plug is configured for moving between said closed position and said open position when button is pressed.

10. The sanitary catheter drain device of claim 9 wherein said button is an integral portion of said plug extending from said housing.

11. The sanitary catheter drain device of claim 9, further comprising a biasing member positioned in said housing, said biasing member biasing said plug such that said drain valve is in said closed position.

12. The sanitary catheter drain device of claim 1, further comprising:
a balloon coupled to said catheter tube adjacent to said insertion end wherein said balloon is configured for positioning in a bladder of the user to inhibit the insertion end from coming out of the bladder of the user; and
a sponge coupled to said catheter tube, said sponge being spaced from said balloon such that said sponge is configured for positioning adjacent to a urethra of the user while the balloon is positioned against a wall of a bladder of the user wherein the catheter tube is configured to be restricted in movement relative to the user.

13. A sanitary catheter drain device comprising:
a catheter tube having an insertion end configured for insertion into a user to establish fluid communication with a bladder of the user, said catheter tube having an open end opposite said insertion end;
a urine collection bag, said urine collection bag having an inlet port and an outlet port, said inlet port being in fluid communication with said open end of said catheter tube; and
a cap, said cap being couplable to said outlet port such that said cap covers said outlet port of said urine collection bag.

14. The sanitary catheter drain device of claim 13, further comprising a pad, said pad being coupled to said cap wherein said pad is positioned to abut said outlet of said drain valve when said cap is engaged to said drain valve.

15. The sanitary catheter drain device of claim 14, further comprising said pad being constructed of an antimicrobial material.

16. The sanitary catheter drain device of claim 14, further comprising said pad having a bottom wall and a peripheral wall coupled to and extending upwardly from a peripheral edge of said bottom wall wherein said pad forms a well in said pad over said bottom wall.

17. The sanitary catheter drain device of claim 16, further comprising said peripheral wall tapering from said bottom wall of said pad to a distal edge of said peripheral wall relative to said bottom wall of said pad.

18. The sanitary catheter drain device of claim 17, further comprising said pad having a height from a bottom edge of said pad to said distal edge of said peripheral wall less than half a length of said cap wherein said pad is positioned in a bottom half of said cap when fully inserted into said cap.

19. The sanitary catheter drain device of claim 16, further comprising said bottom wall having a thickness being greater than twice a maximum thickness of the peripheral wall of said pad.
